(19) United States
(12) Patent Application Publication
(10) Pub. No.: US 2023/0337642 A1
(43) Pub. Date: Oct. 26, 2023

(54) METHOD FOR PRODUCING AVATAR MOUSE AS ATOPIC DERMATITIS ANIMAL MODEL AND USE THEREOF

(71) Applicant: INDUSTRY-ACADEMIC COOPERATION FOUNDATION, YONSEI UNIVERSITY, Seoul (KR)

(72) Inventors: Chang Ook Park, Seoul (KR); Hye Li Kim, Gyeonggi-do (KR)

(21) Appl. No.: 17/904,023

(22) PCT Filed: Feb. 9, 2021
(86) PCT No.: PCT/KR2021/001678

§ 371 Date: Aug. 11, 2022

(30) Foreign Application Priority Data
Feb. 11, 2020 (KR) 10-2020-0016153

(51) Int. Cl. A01K 67/027; A61K 49/00

(52) U.S. Cl. CPC A01K 67/0271; A61K 49/0008; A01K 2227/105; A01K 2267/0368; A01K 2207/12

(57) ABSTRACT

The present invention relates to a method for producing an avatar mouse, which is an animal model of atopic dermatitis, and a method of screening a therapeutic agent for atopic dermatitis using the same. According to the present invention, it is possible to more effectively identify a patient-specific immune response by representing an immune response in an actual atopic dermatitis patient. Thus, the present invention is expected to be widely used in the development of a new immunotherapy system enabling tailored treatment.

### CROSS-REFERENCE TO RELATED APPLICATIONS

This application is a U.S. National Stage application of International Patent Application Publication no. PCT/KR2021/01678, filed Feb. 9, 2021, which claims the benefit of priority of Korean Patent Application no. 10-2020-0016153, filed Feb. 11, 2020.

### TECHNICAL FIELD

The present invention relates to a method for generating an avatar mouse, which is an animal model of atopic dermatitis, and a method of screening a therapeutic agent for atopic dermatitis using the same.

### BACKGROUND ART

Atopic dermatitis is a long-lasting chronic dermatitis, is characterized by the occurrence of extremely itchy eczema lesions on the skin, and can be often seen in people with a history or family history of atopic disease. Atopic dermatitis can be characterized in that, if symptoms thereof occur, the patient scratches or rubs the symptom area, resulting in worsening skin symptoms. The number of patients with atopic dermatitis is increasing worldwide. By the 1970s, about 3% of children under 6 years of age were reported to have atopic dermatitis. However, in recent years, it has been estimated that about 20% of children and about 1 to 3% of adults have atopic dermatitis. In the case of Korea, a survey conducted by Seoul Metropolitan Government in 2008 showed that the prevalence of atopic dermatitis was 19.1% in the questionnaire and 9.2% in the doctor's actual examination, and varied depending on region, age, sex, and socio-cultural characteristics.

It is known that atopic dermatitis is caused by multiple factors, such as a patient's genetic predisposition and environmental factors, immunological abnormalities in patients, and abnormalities in skin barrier function. Atopic dermatitis may cause unbearable itching, which may lead to insomnia, emotional disorders, learning disabilities, reduced ability to adapt to the environment, reduced social activity, and the like. In addition, atopic dermatitis may be also accompanied by severe itching and eczema, which can be mistaken as if the patient is not keeping the skin clean or is suffering from an infectious skin disease, resulting in poor interpersonal relationships. In particular, atopic dermatitis can have a bad effect on self-formation in adolescent patients. Thus, prevention and treatment of atopic dermatitis is essential.

Atopic dermatitis is a chronic disease that is alleviated during treatment and recurs when treatment is stopped, and the causes or exacerbating factors of atopic dermatitis slightly differ between patients. Thus, it is most important to select a treatment method suitable for each patient.

Accordingly, recent studies on patient-tailored preventive or therapeutic agents for more effectively preventing or treating atopic dermatitis depending on the cause thereof have been actively conducted. In these studies, clinical trials are essential, and in these clinical trial procedures, there may be various risk factors, ethical issues may arise, and thus various animal models are used in order to overcome them. As an example, a humanized immunodeficient mouse model is used as a host into which human skin and immune cells have been engrafted and which lack neutrophils, in order to examine the immune response in human skin (European Patent Application Publication No. EP 3556205). However, since atopic dermatitis is a disease that occurs on the skin due to hypersensitivity of the immune system, it should be distinguished from simply examining the immune response in the skin tissue. An example of another mouse model is a patient-derived tumor xenograft humanized mouse model for cancer research (Cancer Discov. 2014 September; 4(9):998-1013.), but atopic dermatitis differs from cancer-related diseases in that a complex immune mechanism needs to be elucidated. As defined by the Korea Centers for Disease Control and Prevention, atopic dermatitis is a chronic allergic inflammatory disease that occurs on the skin, and has the characteristics of both an allergic disease and an inflammatory disease. Since the allergic disease and the inflammatory disease act by different mechanisms, the pathogenesis of each disease needs to be clearly elucidated. Currently, there is no NSG mouse model that represents the immune status of atopic dermatitis patients who are not normal people, and it is also not easy to generate a humanized animal model that perfectly mimics the immunological characteristics of atopic dermatitis. The present inventors have invented a mouse model that represents the immune response of an actual atopic dermatitis patient in order to screen a therapeutic or preventive agent or a cosmetic composition for atopic dermatitis. This mouse model can perfectly reflect a specific immune response, and thus is expected to enable tailored treatment of atopic dermatitis in the near future.

### DISCLOSURE

### Technical Problem

An object of the present invention is to provide an avatar mouse that represents a specific immune response in a patient with a house dust mite (Dermatophagoides farinae)-induced atopic dermatitis.

Another object of the present invention is to provide a method for generating the avatar mouse capable of representing a Dermatophagoides farinae-specific immune response.

Still another object of the present invention is to provide a method of screening a therapeutic or preventive agent or a cosmetic composition for atopic dermatitis using the avatar mouse capable of representing a Dermatophagoides farinae-specific immune response.

However, objects to be achieved by the present invention are not limited to the objects mentioned above, and other objects not mentioned herein will be clearly understood by those of ordinary skill in the art from the following description.

### Technical Solution

Hereinafter, various embodiments described herein will be described with reference to figures. In the following description, numerous specific details are set forth, such as specific configurations, compositions, and processes, etc., in order to provide a thorough understanding of the present invention. However, certain embodiments may be practiced without one or more of these specific details, or in combination with other known methods and configurations. In other instances, known processes and preparation techniques have not been described in particular detail in order to not unnecessarily obscure the present invention. Reference throughout this specification to “one embodiment” or “an embodiment” means that a particular feature, configuration, composition, or characteristic described in connection with the embodiment is included in at least one embodiment of the present invention. Thus, the appearances of the phrase “in one embodiment” or “an embodiment” in various places throughout this specification are not necessarily referring to the same embodiment of the present invention. Additionally, the particular features, configurations, compositions, or characteristics may be combined in any suitable manner in one or more embodiments.

Unless otherwise stated in the specification, all the scientific and technical terms used in the specification have the same meanings as commonly understood by those skilled in the technical field to which the present invention pertains.

According to one embodiment of the present invention, there is provided an avatar mouse which is an animal model of atopic dermatitis obtained by injecting peripheral blood mononuclear cells (PBMCs) derived from atopic dermatitis patients into an immunodeficient mouse. As used herein, the term “peripheral blood mononuclear cells” may be used interchangeably with the term “PBMCs”.

In one embodiment of the present invention, the term “avatar mouse” refers to a patient-tailored animal model, and more specifically, refers to a patient-tailored animal model (xenograft) generated by stereotactically xenografting patient-derived cells or tissue into an immunodeficient animal. The avatar mouse may provide conditions that perfectly reflect the genetic, physiological and environmental characteristics of a patient with a specific disease.

In one embodiment of the present invention, the term “immunodeficient animal” refers to an animal model generated by artificially damaging some components of the immune system at the genetic level so that the immune system may not normally act and a specific disease may occur. As the immunodeficient animal, an animal having a nervous system may be used. Preferably, an immunodeficient mammal may be used. More preferably, an immunodeficient rodent such as an immunodeficient mouse, rat, hamster, beaver, guinea pig or nutria may be used. Most preferably, a nude mouse, an NOD scid gamma (NSG) mouse, a non-obese diabetic (NOD) mouse, a severe combined immunodeficiency (SCID) mouse, an NOD-SCID mouse, an NOG (NOD/SCID Il2rg^{-/-}) mouse or the like may be used, without being limited thereto.

In one embodiment of the present invention, examples of the “immunodeficient mouse” include a nude mouse, an NOD scid gamma (NSG) mouse, a non-obese diabetic (NOD) mouse, a severe combined immunodeficiency (SCID) mouse, an NOD-SCID mouse, an NOG (NOD/SCID Il2rg^{-/-}) mouse, an NSG mouse, and the like. Among them, the first developed nude mouse was known in the 1960s, and since the athymic mouse was known to have weak immune rejection due to the lack of activated T-lymphocytes, studies have been actively conducted on a method of transplanting cancer cells into the mouse. The severe combined immunodeficiency (SCID) mouse was reported by Bosma in the 1980s. The SCID mouse does not reject human cells due to functional deficiency of B cells and T cells, and thus has been widely used in various fields, including cancer, immune diseases, organ transplantation, or infection. The NOD mouse is a mouse deficient in innate immunity, and the SCID mouse is a mouse deficient in acquired immunity, and a mouse model derived from these mice is the NOD-SCID mouse. The NOD-SCID mouse represents the functional characteristics (phenotypes) of the NOD mouse, such as decreased activity of NK (natural killer) cells, the cytokine secretion dysfunction of monocytes, the lack of complement 5 (C5) activity, and at the same time, represents the functional characteristics (phenotypes) of the SCID mouse, such as functional deficiency of B cells and T cells. The NSG mouse refers to an immunodeficient mouse lacking mature T cells, B cells and natural killer (NK) cells. NSG mice developed and sold by Jackson Laboratory are new mice obtained by artificially regulating the cytokine pathway and crossing an NOD-SCID mouse with an Il2rg^{null} mouse (Il2rg-null mouse) in order to further reduce mouse immunity. The NSG mice enable sophisticated modeling in many areas of biology and disease, and thus have been used in many recent studies.

In the present invention, the immunodeficient mouse may be any one selected from the group consisting of a nude mouse, an NOD scid gamma (NSG) mouse, a non-obese diabetic (NOD) mouse, a severe combined immunodeficiency (SCID) mouse, an NOD-SCID mouse, and an NOD/SCID Il2rg^{-/-} (NOG) mouse, and more preferably, may be an NSG mouse. However, the immunodeficient mouse is not limited thereto and may be any mouse belonging to one type of immunodeficient mice, like the above-listed mice.

A disease to be prevented or treated by the present invention may be an atopic disease, specifically, atopic dermatitis. More preferably, the disease may be atopic dermatitis that has been or can be caused by house dust mites (Dermatophagoides farinae), but is not limited thereto.

The animal model mouse of the present invention is characterized by reflecting a specific immune response in an atopic dermatitis patient, more specifically, a Dermatophagoides farinae-sensitized atopic dermatitis patient.

In one embodiment of the present invention, as defined by the Korea Centers for Disease Control and Prevention, the term “atopic dermatitis” refers to a chronic allergic inflammatory skin disease which is common in children and whose symptoms may persist into adulthood. If there are patients with other allergic diseases such as asthma and allergic rhinitis in the family, atopic dermatitis is highly likely to occur, and may be caused by allergens such as food or Dermatophagoides farina. In addition, sudden changes in room temperature and humidity, sweat or saliva, tight or rough clothes, rubbing or scratching the skin, stress, bacterial infection, etc. are also known as aggravating factors for atopic dermatitis. The causative agent or aggravating factor of atopic dermatitis may be determined using a skin test or blood test, a treatment drug or method for atopic dermatitis is applied differently depending on the cause and aggravating factor, and thus it is essential to develop a therapeutic agent by elucidating the cause. Atopic dermatitis has the characteristics of both an allergic disease and an inflammatory disease, and the allergic disease and the inflammatory disease correspond to medically completely different indications. Thus, in the medical world, anti-allergic drugs and non-steroidal anti-inflammatory drugs are distinguished from each other, and non-steroidal anti-inflammatory drugs are not used in the treatment of allergic disease.

In the present invention, regarding the allergic reaction, allergens (foreign antigens) that cause allergy enter our body and activate antigen-specific Th2 cells, and B cells are converted into IgE antibody-producing B cells by cytokines secreted by the activated Th2 cells. When the production of IgE antibody is induced and the antibody is secreted, the secreted IgE antibody binds to mast cells to induce sensitization of mast cells. At this time, as allergens entering from the outside again bind to IgE bound to the mast cell surface, the mast cells are activated to secrete various chemical allergenic mediators such as histamine and inflammatory cytokines, and allergic symptoms begin to appear. When infiltration of eosinophils in the periphery and tissues is induced and enhanced by cytokines (IL-5, etc.) secreted from Th2 cells and mast cells, eosinophilia is induced and eosinophilic inflammation occurs. This is called eosinophilic allergy. Due to eosinophilia as a direct cause, IL-13 induces allergic hypersensitivity, resulting in hypersensitivity. Thus, in order to exhibit an anti-allergic effect, it is possible to use a method of suppressing an enhanced Th2 immune response in allergic disease, or suppressing IgE antibody production in B cells, or inhibiting the action of chemical allergenic mediators such as histamine.

In the present invention, the inflammatory response refers to an external symptom occurring through an immune response caused by the immune system, that is, an innate immune cell recognizing an external antigen or an internal antigen. In this case, the inflammatory response that occurs in the initial immune response by recognizing the external and internal antigens corresponds to an acute inflammatory response, and if the immune response continues to occur due to the constant presence of the antigen, it corresponds to a chronic inflammatory response. An anti-inflammatory agent that suppresses the inflammatory response suppresses the acute inflammatory response along with suppression of the chronic inflammatory response, and also suppresses the immune response of improving resistance to infection, thereby increasing the risk of contracting an infection, whereas an immune enhancer enhances the immune response of improving resistance to infection, but also enhances the inflammatory response, thereby exacerbating the chronic inflammatory response.

In the present invention, regarding atopic dermatitis, “2015 Korean Atopic Dermatitis Treatment Guidelines” published by the Korean Society for Atopic Dermatitis in 2015 proposes “antihistamines, topical calcineurin inhibitors, and topical steroids” for mild atopic dermatitis, and topical calcineurin inhibitors are substances that inhibit T cell activation. In addition, it proposes “cyclosporine, short-term systemic steroids, other systemic immunomodulators (AZA, MMF, MTX, IFN-γ, alitretinoin), phototherapy, antigen-specific immunotherapy, and biological agents” for systemic treatment of moderate and severe atopic dermatitis. Non-steroidal anti-inflammatory drugs are not mentioned anywhere, suggesting that non-steroidal anti-inflammatory drugs are not used for the treatment of atopic dermatitis. This is because atopic dermatitis is not a simple inflammatory response, but is an immune disease caused by immune hypersensitivity. Since most symptoms of atopic dermatitis appear on the skin, it can be misunderstood as a simple skin disease, but atopic dermatitis is a disease to which complex immune mechanisms such as abnormalities in the skin barrier as well as abnormalities in an over-activated immune system are applied (J Interferon Cytokine Res. 2002 April; 22(4):407-12.). Furthermore, the immune response also varies depending on the atopic dermatitis-inducing factors shown in Table 1 below (J Invest Dermatol. 2013 February; 133(2):303-15.). The present invention relates to a humanized animal model of atopic dermatitis in which house dust mites act as a cause of atopic dermatitis. The humanized animal model may represent a specific immune response in atopic dermatitis caused by house dust mites, and thus may be used for screening a tailored therapeutic agent for each patient.

TABLE 1
Factors inducing/exacerbating atopic dermatitis

| | |
|---|---|
| Inhaled antigens | Animal hair, cockroaches, house dust mites, |
| | mold, pollen, dandruff |
| Weather | Sweating, winter, UV rays |
| Psychological stress | — |
| Endocrine | Menstruation, pregnancy |
| Food antigens | Milk, eggs, fish, peanuts, soybeans, flour |
| Contact antigens | Nickel, cobalt, chrome |
| Stimuli | Hot water, soap, tobacco, detergent, |
| | synthetic fiber, wool |
| Microorganisms | Bacteria (Aureus, Streptococcus), fungi, |
| | virus (herpes simplex, water wart) |

In one embodiment of the present invention, the “house dust mite (HDM)” is a type of mite belonging to the family Pyroglyphidae of the order Sarcoptiformes, and is regarded as one of mites in the West. The body of the house dust mite is very small, usually about 0.4 mm in length and 0.3 mm in width, so that it is barely visible when placed on a black background. The body consists of cuticles, and has eight legs, like most mites. Allergens produced by house dust mites are the most common cause of asthma, but also correspond to one of the causes of atopic dermatitis. In particular, for atopic dermatitis caused by house dust mites, there are preventive methods such as maintaining indoor humidity at 40 to 50% and avoiding the use of carpets, bed mattresses, cloth sofas, curtains, etc., where mites habitat. However, in spite of these preventive methods, for appropriate treatment for atopic dermatitis patients who develop a specific immune response to the house dust mite antigen, a mouse model that reflects this immune response is essential. If the mouse model described above is used, it may contribute to discovering a preventive or therapeutic agent or a cosmetic composition for atopic dermatitis caused by house dust mites.

In one embodiment of the present invention, the term “peripheral blood mononuclear cells (PBMCs)” refers to peripheral blood cells that are composed of lymphocytes (T cells, B cells, NK cells) and monocytes and have a round nucleus, among white blood cells contained in human peripheral blood. PBMCs may be extracted and cultured in vitro, and are often used in immunology, transplant immunology, hematology, development of vaccines for malignant tumors and infectious diseases, and high-throughput screening for drug discovery, and research in the fields of biology and chemistry. In addition, human blood has disadvantages in that PBMCs should be isolated within 12 to 24 hours after blood collection in order to prevent cell death, and in that blood supply is not easy due to official procedures such as IRB (Institutional Review Board) approval. In the present invention, PBMCs may be separated into CD3+ T cells, CD3 depleted PBMCs, CD14+ monocyte cells and dendritic cells, and injected into immunodeficient mice.

In one embodiment of the present invention, the PBMCs that are to be injected into the avatar mouse may be separated into CD3+ T cells, CD3 depleted PBMCs, CD14+ monocyte cells or dendritic cells, and injected through different routes.

In the present invention, the “injection” refers to parenteral administration excluding oral administration, and includes injection or infusion techniques such as subcutaneous injection, intradermal injection, intramuscular injection, intraperitoneal injection, intracerebral injection, injection into the sublingual vein, intravenous injection, injection into the penile vein, injection into the caudal vein, and injection into the dorsal metatarsal vein. In addition, the process of injecting PBMCs, more preferably CD3+ T cells, into the mouse of the present invention, may be performed by intravenous injection, and CD3+ T cell-depleted PBMCs (CD3-depleted PBMCs), CD14+ monocyte cells or dendritic cells may be injected through intradermal injection.

The amount of PBMCs injected in the present invention may vary depending on various factors including activity, body weight, administration time, administration route, and excretion rate, and the amount of PBMCs injected may vary depending on mouse's status, body weight, disease severity, drug form, and administration route and period, but may be appropriately selected by those skilled in the art. Administration may be done once or several times. The amount of PBMCs administered does not limit the scope of the present invention in any way.

In one embodiment of the invention, cell surface markers (e.g., human CD3, human CD45, human CD4, and human CD8) that are markers of human T cell activation, and/or cell surface markers (e.g., mouse CD45) that are markers of the functional loss of human T cells, may be analyzed by flow cytometry. The flow cytometry may be performed for analysis or evaluation according to a method well known in the art.

In one embodiment of the present invention, detecting binding between a target analyte present in a blood sample and a binding partner may be achieved by any of various methods known in the art, and this method may comprise a step of detecting a detectable label directly or indirectly attached to the target analyte or the binding partner. The term “detectable label” refers to a material capable of producing a signal indicative of the presence of the detectable label by any appropriate method, and examples of this method include spectroscopic, optical, photochemical, biochemical, enzymatic, electrical and/or immunochemical methods. Examples of detectable labels include a fluorescent moiety, a chemiluminescent moiety, a bioluminescent moiety, an electron dense particle, a magnetic particle, an enzyme, a substrate, a radioisotope and a chromophore. The identity of a particular detectable label or labels used depends on the detection process used. Such detection processes are incorporated in particular assay formats including enzyme linked immunosorbent assay (ELISA), Western blot, immunoprecipitation, immunocytochemistry, immuno-fluorescence assay, liquid chromatography, flow cytometry, other detection processes known in the art, or combinations thereof.

In one embodiment of the present invention, when the cells are positive for human CD3, human CD45, human CD4, or human CD8, the mouse may be considered humanized, and when the cells are positive for mouse CD45, the mouse may be considered non-humanized. However, as an antibody that may be used as a marker for flow cytometry, any antibody may be used without limitation as long as it may be commonly used in the art. By determining whether mice have been humanized by flow cytometry, an avatar mouse that represents an immune response specific to house dust mites according to the present invention may be selected and used for drug or cosmetic screening.

According to another embodiment of the present invention, there is provided a method for generating an avatar mouse which is a humanized mouse model, the method comprising steps of: (a) obtaining a blood sample isolated from an atopic dermatitis patient; (b) obtaining PBMCs from the blood sample; and (c) injecting the obtained PBMCs into an immunodeficient NSG mouse.

More preferably, the generation method according to the present invention may further include isolating PBMCs from a blood sample of an atopic dermatitis patient sensitized to mites and injecting the isolated PBMCs.

The generation method according to the present invention may further comprise a step of obtaining a blood sample isolated from an atopic dermatitis patient having a house dust mite-specific immune response.

The generation method according to the present invention further may comprise a step of separating CD3+ T cells, CD3+ T cell-depleted PBMCs (CD3 depleted PBMCs), CD14+ monocyte cells (CD14+ monocyte cells) or dendritic cells from PBMCs.

The generation method according to the present invention further may comprise a step of injecting CD3+ T cells by intravenous injection and injecting CD3+ T cell-depleted PBMCs (CD3 depleted PBMCs), CD14+ monocyte cells or dendritic cells by intradermal injection.

According to still another embodiment of the present invention, there is provided a method of screening an agent for prevention or treatment of atopic dermatitis or a cosmetic composition for alleviation of atopic dermatitis using the avatar mouse generated by the above-described method.

In one embodiment of the present invention, the term “screening” is one of numerous procedures that are performed during new drug development, and more specifically, refers to a procedure of identifying a candidate substance by discovering active substances and lead substances through screening of compounds selected in basic research. The screening may reduce the time and cost required for new drug development, and may discover an effective new drug substance targeting a selected target disease. For example, examples of the new drug substance include, without limitation, substances that alleviate or beneficially change symptoms of skin disease, more specifically atopic dermatitis disease, upon investigation of new drugs as well as preventive agents or cosmetic compositions.

In the present invention, “tailored medicine” is also referred to as order-made medicine or personalized medicine, and refers to either a method of individually investigating an individual patient's constitution or environment and determining a therapeutic method suitable therefor, or a method of performing treatment.

In the present invention, the term “preventive agent” refers to a candidate substance screened using the avatar mouse of the present invention, and examples of the preventive agent may include, without limitation, any agent that blocks symptoms of atopic dermatitis caused by house dust mites or suppresses or delays dermatitis.

In the present invention, the term “therapeutic agent” refers to a candidate substance screened using the avatar mouse of the present invention, and examples of the therapeutic agent may include, without limitation, any agent that alleviates or beneficially changes skin disease, more specifically atopic dermatitis disease, even more preferably atopic dermatitis disease caused by house dust mites.

In the present invention, the term “cosmetic composition” refers to a candidate substance screened using the avatar mouse of the present invention, and examples of the cosmetic composition may include, without limitation, any agent that alleviates atopic dermatitis symptoms. In addition, the cosmetic composition may be prepared in the form of lotion, nourishing lotion, nourishing essence, massage cream, cosmetic bath water additive, body lotion, body milk, bath oil, baby oil, baby powder, shower gel, shower cream, sunscreen lotion, sunscreen cream, suntan cream, skin lotion, skin cream, UV blocking cosmetics, cleansing milk, anti-hair loss cosmetics, face and body lotion, face and body cream, skin whitening cream, hand lotion, hair lotion, cosmetic cream, jasmine oil, bath soap, water soap, beauty soap, shampoo, hand sanitizer (hand cleaner), medicated soap for non-medical use, cream soap, facial wash, whole body cleanser, scalp cleanser, hair rinse, cosmetic soap, tooth whitening gel, toothpaste, or the like. To this end, the composition of the present invention may further contain a solvent or a suitable carrier, excipient or diluent, which is commonly used in the preparation of a cosmetic composition.

In the present invention, examples of the solvent that may be further contained in the cosmetic composition include, but are not particularly limited to, water, saline, DMSO, or combinations thereof. In addition, examples of the carrier, excipient or diluent include, but are not limited to, purified water, oil, wax, fatty acids, fatty acid alcohols, fatty acid ester, surfactants, humectants, thickeners, antioxidants, viscosity stabilizers, chelating agents, buffer, lower alcohols, and the like. In addition, the cosmetic composition may, if necessary, contain a whitening agent, a moisturizer, a vitamin, a UV blocker, a perfume, a dye, an antibiotic, an antibacterial agent, and an antifungal agent.

In the present invention, examples of the oil include hydrogenated vegetable oil, castor oil, cottonseed oil, olive oil, palm oil, jojoba oil, and avocado oil, and examples of the wax include beeswax, spermaceti, carnauba wax, candelilla wax, montan wax, ceresin, liquid paraffin, and lanolin.

In the present invention, examples of the fatty acid include stearic acid, linoleic acid, linolenic acid, and oleic acid, examples of the fatty acid alcohol include cetyl alcohol, octyl dodecanol, oleyl alcohol, panthenol, lanolin alcohol, stearyl alcohol, and hexadecanol, and examples of the fatty acid ester include isopropyl myristate, isopropyl palmitate, and butyl stearate. Examples of the surfactant include cationic surfactants, anionic surfactants and nonionic surfactants, which are known in the art, and surfactants derived from natural products are preferred. In addition, the cosmetic composition may contain a humectant, a thickener, an antioxidant, etc. which are widely known in the cosmetic field, and the types and amounts thereof are as known in the art.

In one embodiment of the present invention, there is provided a method for generating an atopic dermatitis avatar mouse, the method comprising steps of: (a) separating CD3+ T cells from peripheral blood mononuclear cells (PBMCs) isolated from a subject with atopic dermatitis; and (b) injecting the CD3+ T cells into an immunodeficient mouse. In the method, the atopic dermatitis is atopic dermatitis induced by house dust mites, and the immunodeficient mouse is any one selected from the group consisting of a nude mouse, an NOD scid gamma (NSG) mouse, a non-obese diabetic (NOD) mouse, a severe combined immunodeficiency (SCID) mouse, an NOD-SCID mouse, and an NOG (NOD/SCID Il2rg^{-/-}) mouse. In the method, the injecting in step (b) is intravenous injection, and step (a) further comprises separating CD3 T cell-depleted peripheral blood mononuclear cells (CD3 depleted PBMC), CD14+ monocyte cells or dendritic cells from the peripheral blood mononuclear cells (PBMCs). In the method, step (b) further comprises injecting CD3+ T cell-depleted peripheral blood mononuclear cells or injecting CD14+ monocyte cells and dendritic cells. In addition, injection of the CD3+ T cell-depleted peripheral blood mononuclear cells or injection of the CD14+ monocyte cells and the dendritic cells is performed by intradermal injection.

In another embodiment of the present invention, there is provided an atopic dermatitis avatar mouse generated by the above-described method.

In still another embodiment of the present invention, there is provided a method for screening a substance for treating or alleviating atopic dermatitis, the method comprising steps of: (a) preparing a first subject and a second subject as the avatar mice with atopic dermatitis, (b) treating the first subject with a candidate substance for treating or alleviating atopic dermatitis; and (c) determining that the candidate substance is a substance for treating or alleviating atopic dermatitis, when atopic dermatitis in the first subject has been alleviated compared to that in the second subject. In the method, the atopic dermatitis is atopic dermatitis induced by house dust mites, and the candidate substance is a pharmaceutical composition for preventing or treating atopic dermatitis. In the method, the candidate substance is a cosmetic composition for preventing or alleviating atopic dermatitis, and the cosmetic composition is any one selected from the group consisting of lotion, nourishing lotion, nourishing essence, massage cream, cosmetic bath water additive, body lotion, body milk, bath oil, baby oil, baby powder, shower gel, shower cream, sunscreen lotion, sunscreen cream, suntan cream, skin lotion, skin cream, UV blocking cosmetics, cleansing milk, anti-hair loss cosmetics, face and body lotion, face and body cream, skin whitening cream, hand lotion, hair lotion, cosmetic cream, jasmine oil, bath soap, water soap, beauty soap, shampoo, hand sanitizer (hand cleaner), medicated soap for non-medical use, cream soap, facial wash, whole body cleanser, scalp cleanser, hair rinse, cosmetic soap, tooth whitening gel, and toothpaste.

### Advantageous Effects

The present invention relates to a method for generating an atopic dermatitis avatar mouse, which is an atopic dermatitis animal model that represents the immune status of an atopic dermatitis patient in an NGS mouse and reflects a specific immune response caused by house dust mites, and a method of screening an atopic dermatitis therapeutic agent using the same. The present invention represents an immune response in an actual atopic dermatitis patient, and thus when the present invention is used, it is possible to effectively identify a patient-specific immune response. Accordingly, the present invention may be widely used in the development of a patient-tailored preventive or therapeutic agent depending on the cause of atopic dermatitis.

### BRIEF DESCRIPTION OF DRAWINGS

FIGS. 1A, 1B, 1C, 1D and 1E show the results of examining the engraftment rate of human peripheral blood mononuclear cells (PBMCs) in avatar mice.

FIGS. 2A, 2B, 2C and 2D show the results of analyzing human T cells in the skin lymph nodes, spleens, and skin lesion tissues of avatar mice by flow cytometry.

FIGS. 3A, 3B and 3C show the results of analyzing human T cells and dendritic cells in human PBMCs and the lymph nodes and skin lesion tissues of avatar mice by flow cytometry.

### BEST MODE

One embodiment of the present invention relates to a method for generating an atopic dermatitis avatar mouse, the method comprising steps of: (a) separating CD3+ T cells, CD3+ T cell-depleted peripheral blood mononuclear cells (CD3 depleted PBMCs), CD14+ monocyte cells or dendritic cells from peripheral blood mononuclear cells (PBMCs) isolated from a subject with atopic dermatitis; and (b) injecting the cells into an immunodeficient mouse.

Another embodiment of the present invention relates to an atopic dermatitis avatar mouse generated by the above method.

Still another embodiment of the present invention relates to a method for screening a substance for treating or alleviating atopic dermatitis, the method comprising: (a) preparing a first subject and a second subject as the avatar mice with atopic dermatitis generated by the above method; (b) treating the first subject with a candidate substance for treating or alleviating atopic dermatitis; and (c) determining that the candidate substance is a substance for treating or alleviating atopic dermatitis, when atopic dermatitis in the first subject has been alleviated compared to that in the second subject.

### Mode for Invention

Hereinafter, the present invention will be described in more detail with reference to examples. These examples are only for illustrating the present invention in more detail, and it will be apparent to those of ordinary skill in the art that the scope of the present invention according to the subject matter of the present invention is not limited by these examples.

### EXAMPLE 1

### Recruitment of Atopic Dermatitis Patient Group and Preparation of Immunodeficient NSG Mice

The present inventors conducted all experiments with the approval of the Institutional Review Board (IRB) of the Yonsei University College of Medicine, and blood samples were obtained from atopic dermatitis patients sensitized to Dermatophagoides farinae (house dust mites (HDM)). In addition, immunodeficient NSG mice were purchased from JAX (USA), and six 6-week-old female mice were used in the experiment. The mice were stabilized in the laboratory animal room at a temperature of 23±2° C. and a humidity of 55±5% with a 12-hr dark/12-hr light cycle for 1 week.

### EXAMPLE 2

### Isolation of Peripheral Blood Mononuclear Cells (PBMCs) from Atopic Dermatitis Patients

In order to obtain human PBMCs (huPBMCs), GE Healthcare's Ficoll-Paque reagent was used. The Ficoll-Paque reagent and the blood sample obtained in Example 1 were placed in a 15-ml tube at a ratio of 1:1 so that they were not mixed together. The tube was placed in a centrifuge, followed by centrifugation at 2,500 rpm and RT for 15 minutes. Red blood cells were collected in the bottom layer and the buffy coat layer and plasma layer were present thereon. The plasma layer was discarded and the buffy coat layer was collected to obtain PBMCs. The obtained PBMCs were transferred to a fresh tube and mixed with an EDTA solution at a ratio of 1:1, and the mixture was placed in centrifuge and centrifuged at 1,500 rpm and RT for 10 minutes. Thereafter, the supernatant was discarded and the remaining pellet (PBMCs) was finally obtained. CD3+ T cells as pathogen-induced cells causing atopic dermatitis were separated from the PBMC. In addition, CD14+ cells as antigen presenting cells were separated from CD3-depleted PBMCs.

To separate the CD3+ T cells, Miltenyi Biotec's Pan T Cell Isolation Kit was used. According to the manufacturer's instructions of the kit, 400 μl MACS buffer per 10⁸ cells was added to the PBMCs, and 100 μl Pan T Cell Biotion Antibody Cocktail per 10⁸ cells was added thereto. Then, the mixture was incubated at 2 to 8° C. for 5 minutes, and then 300 μl MACS buffer per 10⁸ cells was added and 200 μl Pan T Cell MicroBead Cocktail per 10⁸ cells was added, followed by incubation incubated at 2 to 8° C. for 10 minutes. Thereafter, magnetic separation was performed using an LS column to obtain CD3+ T cells and the remaining CD3-depleted PBMCs. In addition, to obtain CD14+ monocyte cells and dendritic cells, the experiment was performed on the CD3 depleted PBMCs obtained in the above experiment using Miltenyi Biotec's CD14 MicroBeads, human kit, or Pan DC Enrichment kit according to the manufacturer's instructions of the kit, thus obtaining CD14+ monocyte cells or dendritic cells. A more detailed experimental method is as follows.

The CD3-depleted PBMCs were centrifuged at 300 g for 10 minutes, and the supernatant was discarded. 80 μl MACS buffer/10⁷ cells was added to and mixed well with the remaining pellet, and 20 μl CD14 MicroBeads per 10⁷ cells were added thereto, followed by incubation at 2 to 8° C. for 15 minutes. Thereafter, the cells were washed with 1 to 2 mL MACS buffer per 10⁷ cells and then centrifuged for 300×g for 10 minutes. The supernatant was discarded, and 500 μl of MACS buffer was added to and mixed well with the remaining pellet, and magnetic separation was performed using an LS column to obtain CD14+ monocyte cells.

To separate dendritic cells, CD3-depleted PBMCs were centrifuged at 300×g for 10 minutes, and then 350 μl MACS buffer per 10⁸ cells were added to and mixed well with the cells, and 50 μl FcR blocking reagent per 10⁸ cells was added to the cells. Next, 100 μl Pan DC Biotin-Antibody Cocktail per 10⁸ cells was added to the cells, followed by incubation at room temperature for 5 minutes. Thereafter, 400 μl MACS buffer per 10⁸ cells were added to the cells and 100 μl Pan DC MicoBead-Antibody Cocktail per 10⁸ cells were added thereto, followed by incubation at room temperature for 5 minutes. The cells were washed with 10 mL MACS buffer/10⁸ cells and centrifuged at 300×g for 10 minutes, and the supernatant was discarded. 500 μl MACS buffer/10⁸ cells was added to and mixed well with the remaining pellet, and magnetic separation was performed using an LS column, thus finally obtaining dendritic cells.

### EXAMPLE 3

### Generation of Avatar Mice Transplanted with huPBMCs by Intravenous Injection and Intradermal Injection

Human PBMC cells obtained from the blood of atopic dermatitis patients by the method of Example 2 were injected into immunodeficient NSG mice by two different routes of administration. 2×10⁶ pathogen-induced CD3+ T cells/20 μg were injected by intravenous injection, and in this case, CD3 T cells as well as whole PBMCs could be used. In order to sensitize intradermal dendritic cells as antigen-presenting cells, 3×10⁶ CD3-depleted PBMCs per 30 μg or 3×10⁶ CD14+ cells per 30 μg and 3×10⁶ dendritic cells per 30 μg were injected into the back of each mouse by intradermal injection. Avatar mice were generated by injecting PBMCs, derived from atopic dermatitis patient sensitized to house dust mites (HDMs), into the immunodeficient NSG mice. The avatar mice are meaningful in that they are mouse models that represent an atopic dermatitis patient-specific immune response using house dust mites, which are major allergens of atopic dermatitis.

After removing the hair on the back of the avatar mouse generated by the above-described method, dead skin cells were removed with 3M Tegaderm film once every 3 days, and 100 mg of a house dust mite ointment was applied to the back. The above procedure was carried out for 4 weeks. The house dust mite ointment used was an atopic dermatitis challenging ointment (Biostir, Osaka, Japan) which is an ointment containing a natural ingredient derived from mites. The ointment may be applied to induce house dust mite-specific allergic rhinitis, allergic asthma, or atopic dermatitis, and was used in the present invention to induce atopic dermatitis.

### EXAMPLE 4

### Flow Cytometry to Determine Human PBMC Engraftment Rate and Identify Humanized Mice

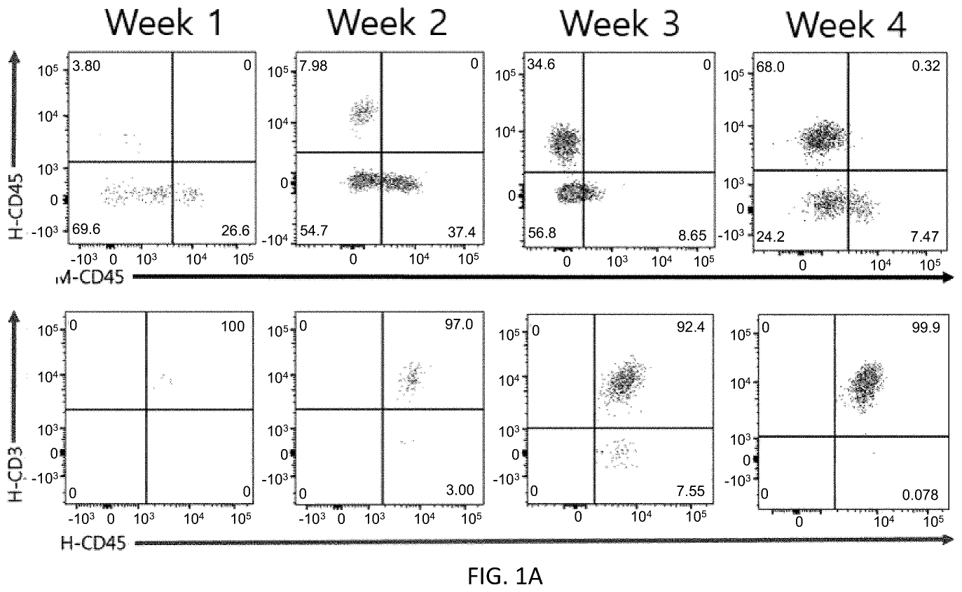

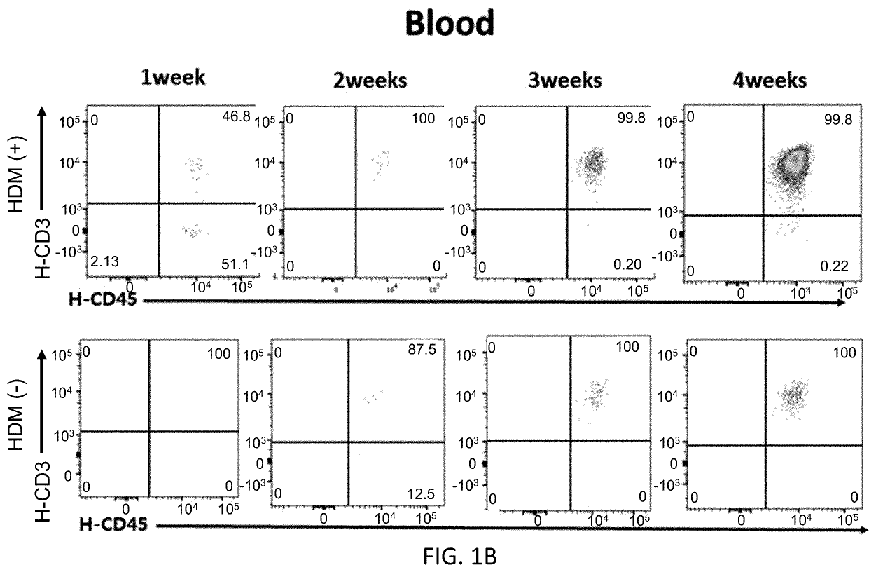

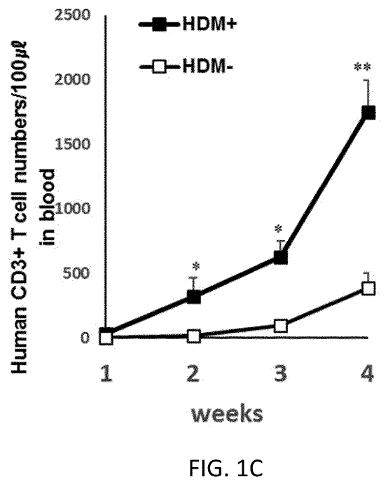

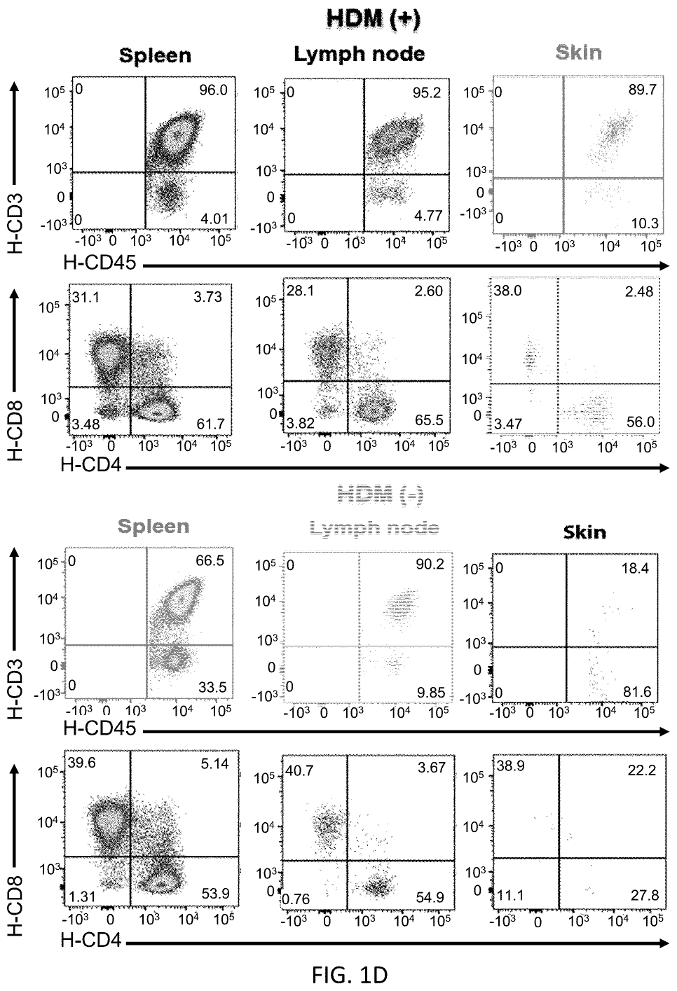

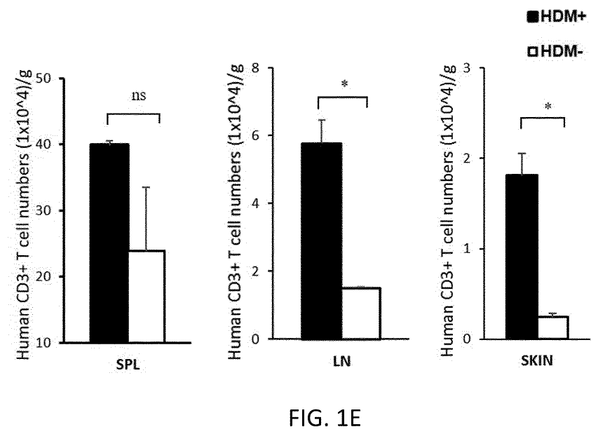

The engraftment rate of human PBMCs in the atopic dermatitis mouse model generated as described in Example 3 was examined over 4 weeks after PBMC injection. In order to confirm whether the avatar mouse was humanized, the results were observed at regular intervals every week. T cells were separated from the immune cells of the mouse animal model by orbital blood sampling, and the separated T cells were dispensed into a FACS tube at a density of 2.0×10⁶ cells/tube, and the content in each tube was washed twice with 1 mL FACS buffer at 100 rpm at 4° C. for 3 minutes. Each antibody to be checked below was mixed with FACS buffer, and 50 μl of the mixture was dispensed into each FACS tube and covered with a foil. After 20 minutes, the content in each tube was washed twice with 1 mL of FACS buffer, and then 0.5 mL of the buffer was placed in the FACS tube and the content in the tube was resuspended. Analysis was performed by flow cytometry. In this case, the cell surface was stained, and then the cells were fixed and permeabilized, and the sample was analyzed by flow cytometry in a Becton Dickinson FACS Canto instrument according to the manufacturer's protocol. The data were analyzed using FACS Diva software (V5.1). The results are shown in FIG. 1A. Referring to FIG. 1A, it was confirmed that the cells were negative for mouse CD45 while being positive for human CD45 over 1 to 4 weeks, and that the cells were positive for both human CD45 and human CD3. The process of changes in the engraftment rate of human PBMC cells over time was confirmed by comparing and confirming the correlation between human CD45 and human CD3 as human T cell markers from the mouse cells and the correlation between human CD45 and mouse CD45.

In addition, FIGS. 1B and 1C are graphs showing the results of analyzing the number of human CD3 and CD45 positive cells over 4 weeks by flow cytometry in order to confirm the engraftment rate of human PBMCs in the avatar mouse blood for each week. Referring to FIGS. 1B and 1C, it could be confirmed that the human T cells were much more in the avatar mouse having an inflammatory response induced using house dust mites than in the avatar mouse not treated with HDMs, and it was confirmed that the human CD3 positive percentage in the avatar mouse treated with house dust mites significantly increased over time, suggesting that the engraftment efficiency in the avatar mouse treated with house dust mites increased. In addition, FIGS. 1D and 1E show the results of analyzing human T cells in the spleens, lymph nodes, and skin lesions of the avatar mouse treated with house dust mites and the avatar mouse not treated with house dust mites. It can be seen that these results are the same as the results obtained in the blood.

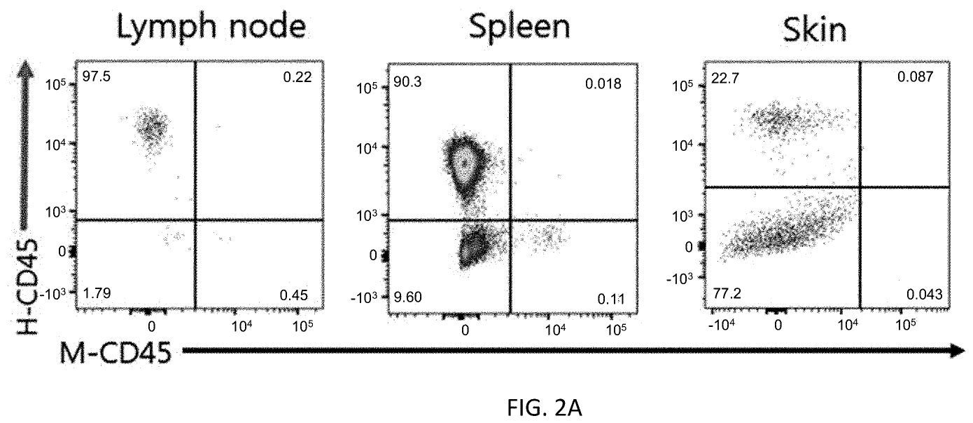

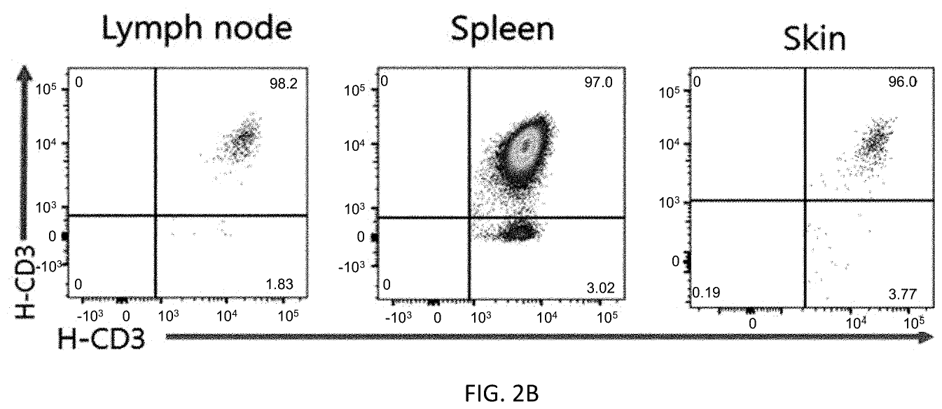

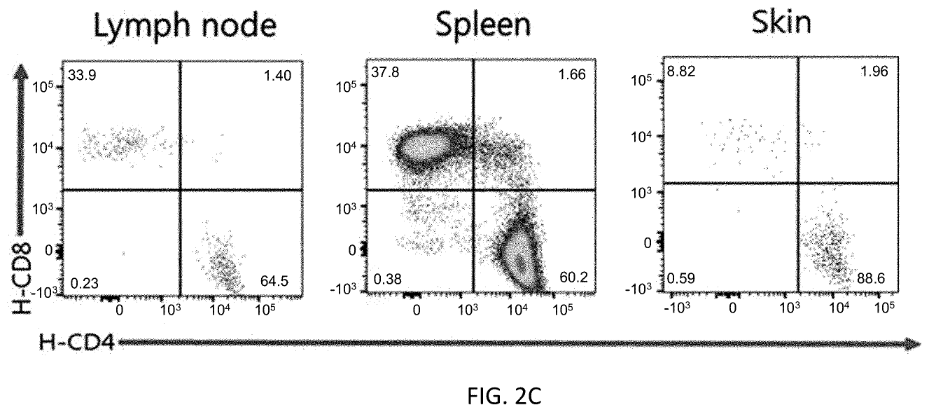

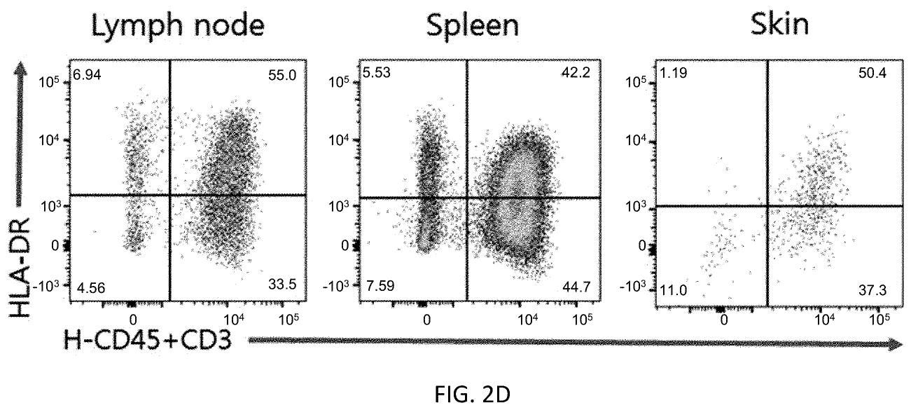

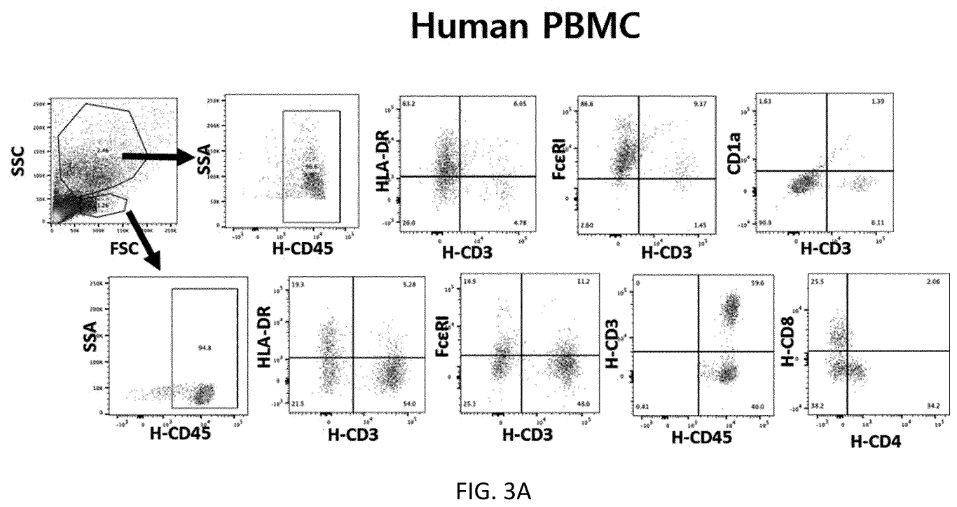

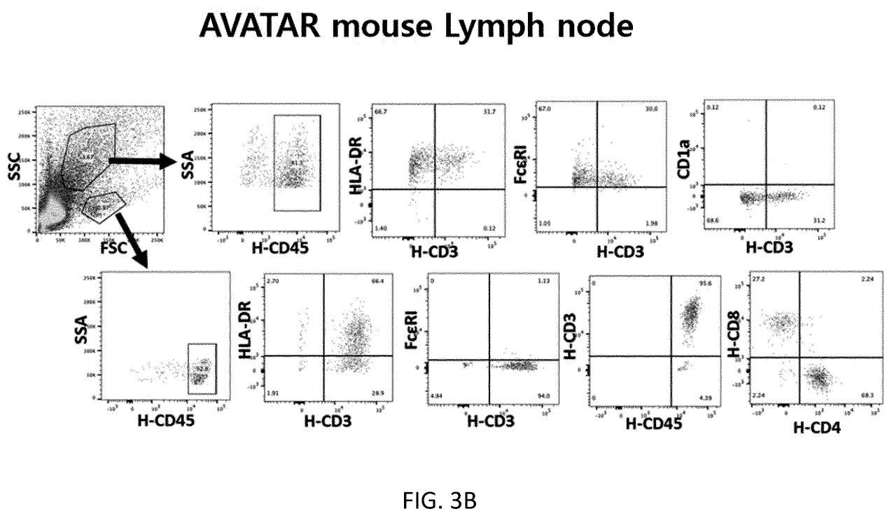

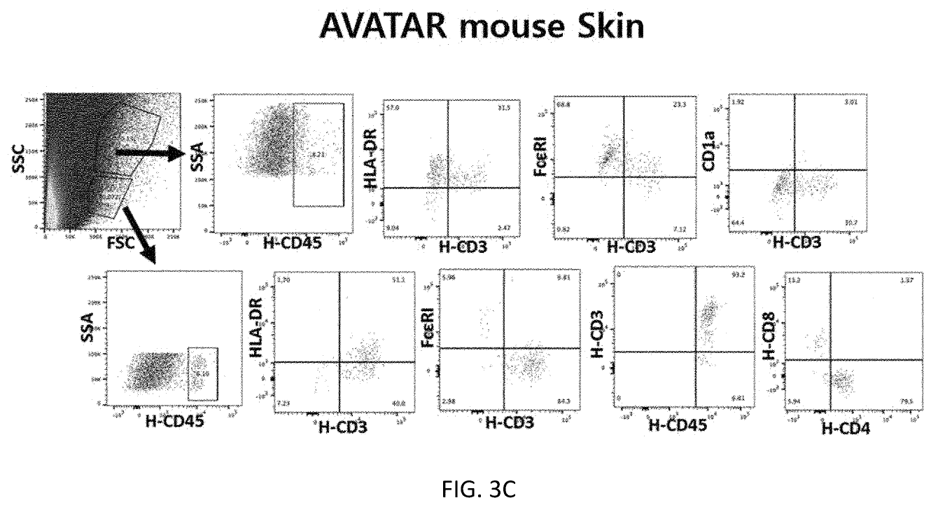

Finally, after 4 weeks, flow cytometry was performed on lymph nodes, spleens, and skin lesion tissues in the same manner as above, and the results are shown in FIGS. 2A, 2B, 2C and 2D. It was confirmed that the lymph nodes were positive for human CD45, CD3, CD4 and CD8, and the same trend was also observed in spleens and skin lesions. As described above, it was confirmed that all the tissues were positive for human markers and negative for mouse CD45 as a mouse marker. In addition, FIGS. 3A, 3B and 3C show the results of analyzing human T cells and human antigen-presenting cells (APCs) in human PBMCs and the lymph nodes and skin lesion tissues of the avatar mouse. In FIGS. 3A, 3B and 3C, the non-lymphocyte portion corresponds to the upper portion in each figure, and the lymphocyte portion corresponds to the lower portion in each figure. As a result of separately analyzing lymphocytes and non-lymphocytes in FSC-SSC of human PBMCs, human T cells were identified in the lymphocytes, and APC cells were identified in the non-lymphocytes. Similarly, as a result of analyzing two divided populations (lymphocytes and non-lymphocytes) in the lymph nodes and skin lesions of the avatar mouse, the expression of cells as in human PBMC was confirmed. Thereby, it was confirmed that a patient-tailored atopic dermatitis animal model, that is, a humanized avatar mouse that represents the patient's immune response, was successfully generated. Only mice confirmed to have these characteristics may be selected and used for screening.

Although the present invention has been described in detail with reference to the specific features, it will be apparent to those skilled in the art that this detailed description is only of a preferred embodiment thereof, and does not limit the scope of the present invention. Thus, the substantial scope of the present invention will be defined by the appended claims and equivalents thereto.

### INDUSTRIAL APPLICABILITY

The present invention is directed to a human T cell-containing atopic dermatitis animal model that represents a specific immune response caused by house dust mites in an actual atopic dermatitis patient. When the present invention is used, it is possible to effectively identify a specific immune response depending on the cause of atomic dermatitis. Accordingly, the present invention may be widely used in the development of a patient-tailored preventive or therapeutic agent for atopic dermatitis.

1. A method for generating an atopic dermatitis avatar mouse, the method comprising steps of:
(a) separating CD3+ T cells from peripheral blood mononuclear cells (PBMCs) isolated from a subject with atopic dermatitis; and
(b) injecting the CD3+ T cells into an immunodeficient mouse.

2. The method of claim 1, wherein the atopic dermatitis is atopic dermatitis induced by house dust mites.

3. The method of claim 1, wherein the immunodeficient mouse is any one selected from the group consisting of a nude mouse, an NOD scid gamma (NSG) mouse, a non-obese diabetic (NOD) mouse, a severe combined immunodeficiency (SCID) mouse, an NOD-SCID mouse, and an NOG (NOD/SCID Il2rg^{-/-}) mouse.

4. The method of claim 1, wherein the injecting in step (b) is performed by intravenous injection.

5. The method of claim 1, wherein step (a) further comprises separating CD3+ T cell-depleted peripheral blood mononuclear cells (CD3 depleted PBMCs), CD14+ monocyte cells or dendritic cells from the peripheral blood mononuclear cells (PBMCs).

6. The method of claim 1, wherein step (b) further comprises injecting CD3+ T cell-depleted peripheral blood mononuclear cells or injecting CD14+ monocyte cells and dendritic cells.

7. The method of claim 6, wherein the injecting of the CD3+ T cell-depleted peripheral blood mononuclear cells or the injecting of the CD14+ monocyte cells and the dendritic cells is performed by intradermal injection.

8. An atopic dermatitis avatar mouse generated by the method of claim 1.

9. A method for screening a substance for treating or alleviating atopic dermatitis, the method comprising steps of:
(a) preparing a first subject and a second subject, each of the first subject and the second subject being a atopic dermatitis avatar mice of claim 8;
(b) treating the first subject with a candidate substance for treating or alleviating atopic dermatitis; and
(c) determining that the candidate substance is a substance for treating or alleviating atopic dermatitis, when atopic dermatitis in the first subject has been alleviated compared to that in the second subject.

10. The method of claim 9, wherein the atopic dermatitis is atopic dermatitis induced by house dust mites.

11. The method of claim 9, wherein the candidate substance is a pharmaceutical composition for preventing or treating atopic dermatitis.

12. The method of claim 9, wherein the candidate substance is a cosmetic composition for preventing or alleviating atopic dermatitis.

13. The method of claim 12, wherein the cosmetic composition is any one selected from the group consisting of lotion, nourishing lotion, nourishing essence, massage cream, cosmetic bath water additive, body lotion, body milk, bath oil, baby oil, baby powder, shower gel, shower cream, sunscreen lotion, sunscreen cream, suntan cream, skin lotion, skin cream, UV blocking cosmetics, cleansing milk, anti-hair loss cosmetics, face and body lotion, face and body cream, skin whitening cream, hand lotion, hair lotion, cosmetic cream, jasmine oil, bath soap, water soap, beauty soap, shampoo, hand sanitizer (hand cleaner), medicated soap for non-medical use, cream soap, facial wash, whole body cleanser, scalp cleanser, hair rinse, cosmetic soap, tooth whitening gel, and toothpaste.
